(19) United States
(12) Patent Application Publication
(10) Pub. No.: US 2017/0105609 A1
(43) Pub. Date: Apr. 20, 2017

(54) SCANNING-TYPE ENDOSCOPE APPARATUS

(71) Applicant: OLYMPUS CORPORATION, Tokyo (JP)

(72) Inventors: Noboru NAKAYAMA, Hachioji-shi, Tokyo (JP); Masanori SUMIYOSHI, Hachioji-shi, Tokyo (JP)

(73) Assignee: OLYMPUS CORPORATION, Tokyo (JP)

(21) Appl. No.: 15/391,066

(22) Filed: Dec. 27, 2016

Related U.S. Application Data
(63) Continuation of application No. PCT/JP2016/053747, filed on Feb. 9, 2016.

(30) Foreign Application Priority Data
Apr. 20, 2015 (JP) 2015-086156

(51) Int. Cl. A61B 1/04; G02B 26/10; H04N 5/225; G02B 23/24; A61B 1/00; A61B 1/07

(52) U.S. Cl. CPC A61B 1/04; A61B 1/00009; A61B 1/00045; A61B 1/07; H04N 5/2256; G02B 23/2469; G02B 26/103; G02B 23/2484; H04N 2005/2255

(57) ABSTRACT

A scanning-type endoscope apparatus includes: a scanning-type endoscope performing scanning with illumination light on a subject by driving an actuator; a storing portion storing drive conditions information and specific information; a reading portion; a controlling portion driving the actuator based on the drive conditions information; a body apparatus; a signal generating portion generating a signal corresponding to light from the subject; and an image information generating portion generating image information based on the signal. The controlling portion causes the image information to be generated using the signal in a state where reading of the drive conditions information is complete but reading of the specific information is not complete, and causes the image information to be generated using the signal and the specific information in a state where reading of the drive conditions information and the specific information is complete.

### CROSS REFERENCE TO RELATED APPLICATION

This application is a continuation application of PCT/JP2016/053747 filed on Feb. 9, 2016 and claims benefit of Japanese Application No. 2015-086156 filed in Japan on Apr. 20, 2015, the entire contents of which are incorporated herein by this reference.

### BACKGROUND OF THE INVENTION

1. Field of the Invention

The present invention relates to a scanning-type endoscope apparatus for performing scanning with illumination light.

2. Description of the Related Art

Recently, endoscopes have been widely used in a medical field and the like. Further, various techniques are proposed in order to reduce a diameter of an insertion portion to be inserted into a subject. As an example of such techniques, there is a scanning-type endoscope apparatus.

For example, a conventional example in Japanese Patent Application Laid-Open Publication No. 2014-90780 discloses that, in such a calibration system that manufacturing cost of a jig for calibrating a trajectory of optical scanning is held down, identification information and information about properties and the like about an endoscope stored in a sub-memory are read, transmitted to a CPU and stored into a CPU memory when the endoscope and a processor are electrically connected at system startup, and the CPU reads out the information when necessary, generates a signal required to control the endoscope and specify set values required for a scanning driver.

### SUMMARY OF THE INVENTION

A scanning-type endoscope apparatus of an aspect of the invention includes: a scanning-type endoscope configured to, by driving an actuator for swinging a fiber configured to guide illumination light radiated to a subject, perform scanning with the illumination light on the subject; a storing portion provided in the scanning-type endoscope and configured to store drive conditions information which is information about drive conditions of the actuator and specific information which is information specific to the scanning-type endoscope, the specific information having a larger amount of data than the drive conditions information and being about the scanning; a reading portion configured to read the drive conditions information and the specific information among pieces of information stored in the storing portion; a controlling portion configured to perform control to drive the actuator based on the drive conditions information read by the reading portion; a body apparatus to which the scanning-type endoscope is detachably connected, the body apparatus comprising the reading portion and the controlling portion; a signal generating portion provided in the body apparatus and configured to swing the fiber using the actuator driven in accordance with the drive conditions information read by the reading portion, receive light from the subject at a time of radiating the illumination light, and generate a photoelectrically converted signal; and an image information generating portion provided in the body apparatus and configured to generate image information corresponding to a radiation position of the illumination light based on the signal. In a first state in which reading of the drive conditions information from the storing portion by the reading portion is complete but reading of the specific information is not complete, the controlling portion controls the image information generating portion to generate the image information to be displayed on a display apparatus using at least the signal generated by the signal generating portion; and, in a second state in which reading of the drive conditions information and the specific information from the storing portion by the reading portion is complete, the controlling portion controls the image information generating portion to generate the image information to be displayed on the display apparatus using both of the signal generated by the signal generating portion and the specific information read by the reading portion.

### BRIEF DESCRIPTION OF THE DRAWINGS

FIG. 1 is a diagram showing a whole configuration of a scanning-type endoscope apparatus of a first embodiment of the present invention;

FIG. 2 is a cross-sectional view showing a configuration of an actuator by a section along an A-A line in FIG. 1;

FIG. 3 is a diagram showing a waveform of a drive signal for driving the actuator;

FIG. 4 is a diagram showing a trajectory obtained by a distal end of an optical fiber being swung by the drive signal of FIG. 3;

FIG. 5 is a diagram showing content of various kinds of data in scope ID data stored in each scanning-type endoscope;

FIG. 6A is a diagram showing details of the various kinds of data of FIG. 5 being associated with addresses and stored in a memory in a tabular format;

FIG. 6B is a diagram showing details of calibration data in FIG. 6A in a tabular format;

FIG. 7 is a diagram showing a configuration of a patient circuit;

FIG. 8 is a flowchart showing a typical process procedure of the first embodiment;

FIG. 9 is a diagram showing timings of the typical process procedure of the first embodiment;

FIG. 10 is a diagram showing an example in which an amount of the calibration data has been reduced; and

FIG. 11 is a flowchart showing a process procedure in a modification of the first embodiment.

### DETAILED DESCRIPTION OF THE PREFERRED EMBODIMENT

An embodiment of the present invention will be described below with reference to drawings.

### First Embodiment

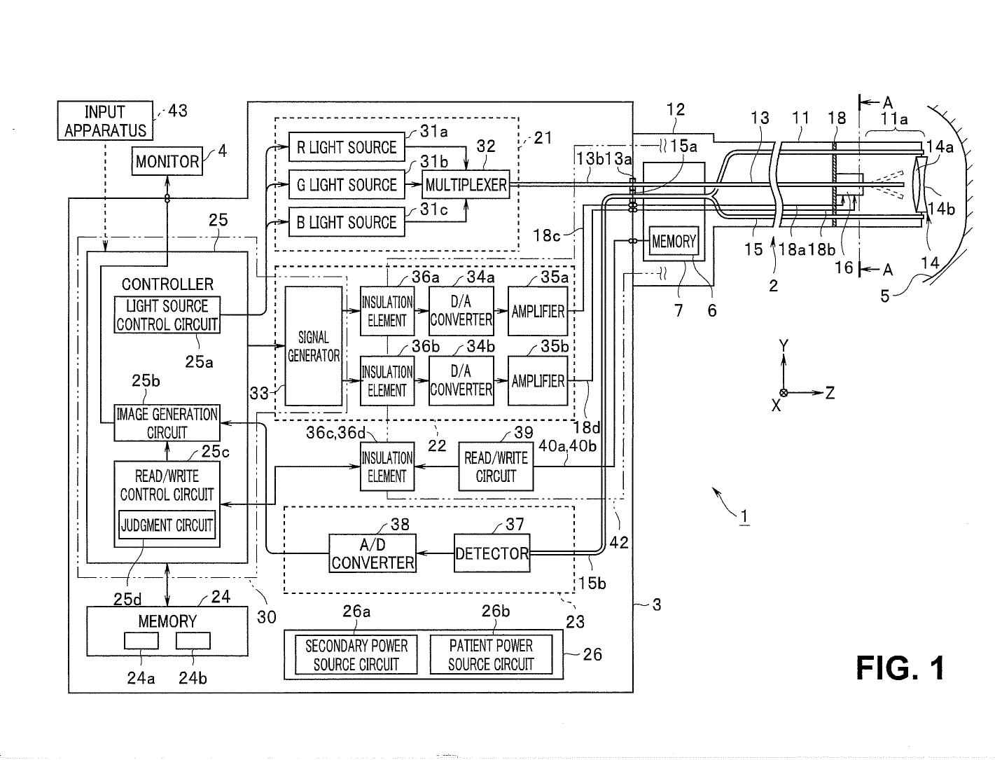

As shown in FIG. 1, a scanning-type endoscope apparatus 1 of an embodiment of the present invention includes a scanning-type endoscope 2 configured to be inserted into a body cavity of a subject 5, a body apparatus (or a scanning-type endoscope control device) 3 to which the scanning-type endoscope 2 is detachably connected, and a monitor 4 as a display apparatus to be connected to the body apparatus 3. The scanning-type endoscope 2 includes a memory 6 in which scope ID data including specific information specific to each scanning-type endoscope 2 is stored, and the memory 6 is provided on a scope substrate 7 provided in the scanning-type endoscope 2.

Further, the scanning-type endoscope 2 includes an insertion portion 11 formed having an elongated shape which can be inserted into a body or a body cavity of the subject 5 and flexibility. On a proximal end portion of the insertion portion 11, a scope connector (simply abbreviated as a connector) 12 for detachably connecting the scanning-type endoscope 2 to the body apparatus 3 is provided. Note that the scope substrate 7 is provided inside the connector 12.

In the insertion portion 11, an illumination optical fiber 13 to be a light guide member configured to guide illumination light supplied from a light source unit 21 of the body apparatus 3 is inserted from the proximal end portion to a distal end portion 11a. Illumination light guided by the illumination optical fiber 13 is emitted from a distal end of the illumination optical fiber 13 toward an object such as an examination site in the subject 5 via a light condensing optical system 14 opposite to the distal end.

Further, inside the insertion portion 11, a light receiving optical fiber 15 configured to receive return light from the subject 5 (an object on the subject 5 side) and guide the return light to a detection unit 23 constituting a detecting portion of the body apparatus 3 is inserted.

An end portion of the illumination optical fiber 13 which includes a light incident surface is connected to a distal end side end portion of an illumination optical fiber 13b provided inside the body apparatus 3, in an optical connector 13a. A proximal end side end portion of the illumination optical fiber 13b which includes an optical incident surface is arranged near a multiplexer 32 inside the light source unit 21.

Further, an end portion of the illumination optical fiber 13 which includes a light emission surface is arranged at a position near and facing the light condensing optical system 14 provided in the distal end portion 11a of the insertion portion 11 and swung by an actuator 16 in that state.

An end portion of the light receiving optical fiber 15 which includes a light incident surface is arranged, for example, along a circle around a light emission surface of the light condensing optical system 14 on a distal end face of the distal end portion 11a of the insertion portion 11. Further, a proximal end side end portion of the light receiving optical fiber 15 to be a light emission surface of the light receiving optical fiber 15 is connected to a distal end side end portion of a light receiving optical fiber 15b provided inside the body apparatus 3, in an optical connector 15a. A proximal end side end portion of the light receiving optical fiber 15b is arranged near a detector 37 in the detection unit 23.

Further, the detection unit 23 is not limited to such that is provided in the body apparatus 3 but may be provided in the scanning-type endoscope 2.

The light condensing optical system 14 forms an optical system having an achromatic function and configured with a convex lens 14a and a concave lens 14b and is configured to condense illumination light from a distal end face of the illumination optical fiber 13 and emits the illumination light to an object side.

At a midway portion of the illumination optical fiber 13, close to the distal end portion 11a in the insertion portion 11, the actuator 16 constituting a scanner for driving a distal end side of the illumination optical fiber 13 in a direction orthogonal to a longitudinal direction of the illumination optical fiber 13 based on a drive signal outputted from a drive unit 22 of the body apparatus 3 is provided. Actuator elements 17a, 17b and 17c, 17d constituting the actuator 16 are connected to drive wires 18a and 18b inserted inside the insertion portion 11. The drive wires 18a and 18b are connected to drive wires 18c and 18d inside the body apparatus 3 via contact points of the connector 12. The drive wires 18c and 18d are connected to the drive unit 22, and a drive signal is applied.

When the longitudinal direction of the illumination optical fiber 13 is assumed to be a Z axis direction, the actuator 16 moves the distal end side of the illumination optical fiber 13 as indicated by dotted lines in FIG. 1 from a state shown by a solid line in FIG. 1 by expanding or contracting in the Z axis direction by application of a drive signal to perform scanning with illumination light emitted from the distal end face of the illumination optical fiber 13 in X and Y axis directions vertical to the Z axis.

In order that it is easy to cause the distal end side of the illumination optical fiber 13 to perform scanning in the X and Y axis directions when the actuator 16 is driven by application of a drive signal, a proximal end side portion of the actuator 16 along the Z axis is fixed to an inner surface of the insertion portion 11 via a supporting member 18.

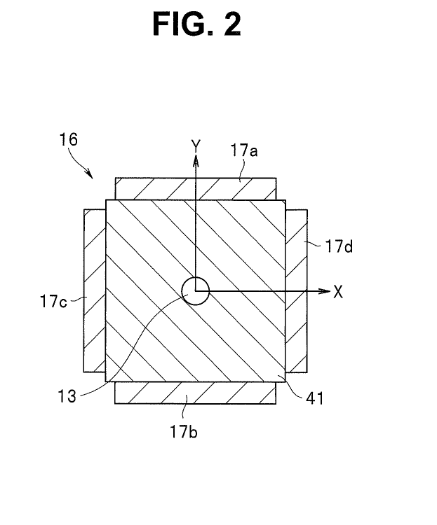

The illumination optical fiber 13 and the actuator 16 are arranged on a section vertical to a longitudinal axis direction of the insertion portion 11, respectively, so as to have a positional relation, for example, as shown in FIG. 2. FIG. 2 is a cross-sectional view showing a configuration of the actuator 16 provided in the scanning-type endoscope 2.

As shown in FIG. 2, a ferrule 41 as a connecting member is arranged between the illumination optical fiber 13 and the actuator 16. More specifically, the ferrule 41 is formed, for example, with zirconia (ceramics) or nickel.

As shown in FIG. 2, the ferrule 41 is formed so as to have a square prism shape, and the illumination optical fiber 13 passing through a hole along a central axis is fixed. On both sides in the Y axis direction (vertical direction of paper) and both sides in the X axis direction (horizontal direction of the paper), the actuator elements 17a, 17b and 17c, 17d which form the actuator 16 are attached.

Each actuator element is configured with a piezoelectric element made of PZT (lead zirconate titanate) or the like, and expands and contracts in a longitudinal direction (in the Z axis direction in FIG. 2) by application of a drive signal. Therefore, for example, by applying drive signals with opposite phases (for causing one actuator to expand and the other to contract) to the actuator elements 17a and 17b in a state that proximal ends are held or fixed, the distal end side of the light receiving optical fiber 15 can be vibrated (swung) in a vertical direction as indicated by the dot lines in FIG. 1.

Note that each of the actuator elements 17a to 17d is polarization-processed so that a polarization direction becomes a predetermined direction, and electrodes (not shown) to which a drive signal is applied are provided on both on opposite faces.

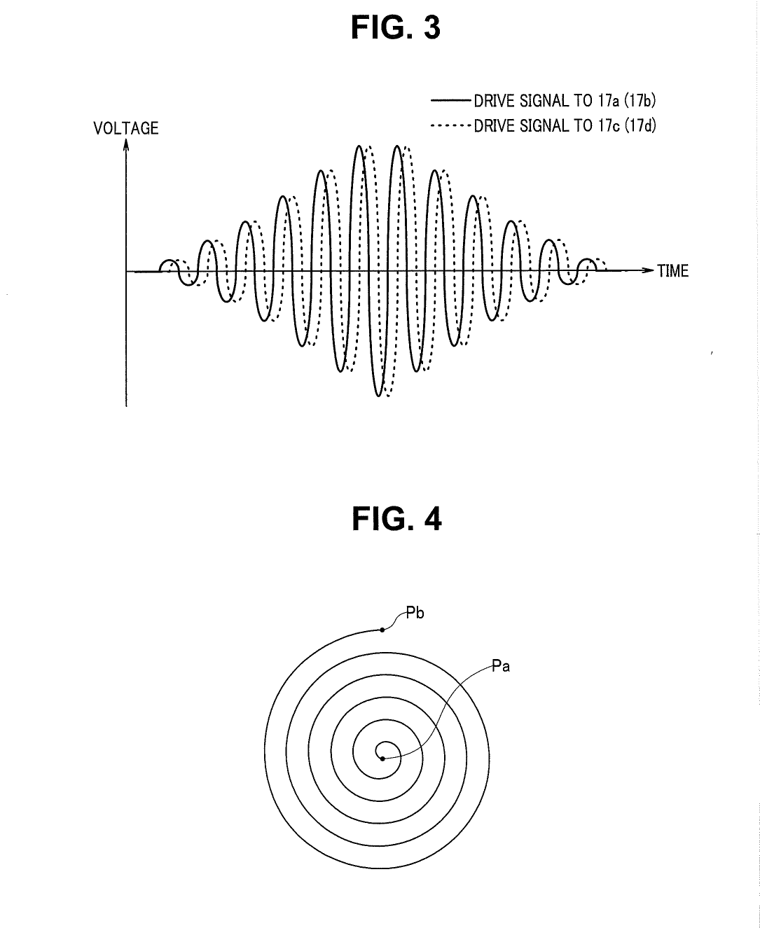

FIG. 3 shows waveforms of drive signals which drive the actuator elements 17a (and 17b) and 17c (and 17d).

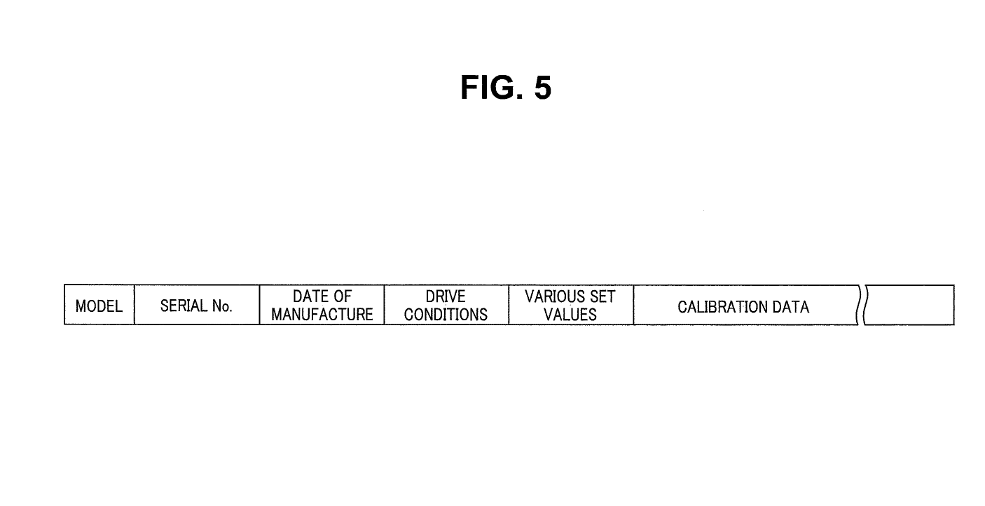

As shown in FIG. 3, the drive signals which drive the actuator elements 17a (and 17b) and 17c (and 17d) are caused to be changed in sine waveform shapes with almost same waveforms obtained by shifting a phase of one drive signal; and voltages (amplitudes) of the signals are caused to gradually increase from a value of 0 corresponding to a scanning start position Pa (see FIG. 4) to a value corresponding to a scanning end position Pb (see FIG. 4) and then caused to gradually decrease again to return to the value of 0 corresponding to the scanning start position Pa.

By applying such drive signals to the actuator element 17a (and 17b) and the actuator element 17c (and 17d), the distal end side of the illumination optical fiber 13 driven by the actuator 16 is moved (caused to scan) in a spiral shape from the scanning start position Pa to the scanning end position Pb as shown in FIG. 4. In response to scanning by the distal end of the illumination optical fiber 13, illumination light emitted from the distal end of the illumination optical fiber 13 also performs optical scanning on a surface of the subject 5 in a spiral shape. Note that the illumination light is controlled so that not continuous light emission but pulsed light emission is performed.

When the actuator 16 is driven by the drive signals in the drive waveforms as shown in FIG. 3, the scanning position shown in FIG. 4 changes actually because of individual difference among electrical characteristics and the like of the actuator elements 17a to 17d forming the actuator 16. Therefore, a two-dimensional coordinate position where light obtained in the case of driving the actuator 16 of each scanning-type endoscope 2 with the drive signals as shown in FIG. 3 is actually radiated to a reference object surface is examined, and such calibration data that a scanning position is calibrated so that the examined proper coordinate position (also referred to as a scanning position or a radiation position) can be acquired is stored in advance in the memory 6 in the scanning-type endoscope 2 equipped with the actuator 16.

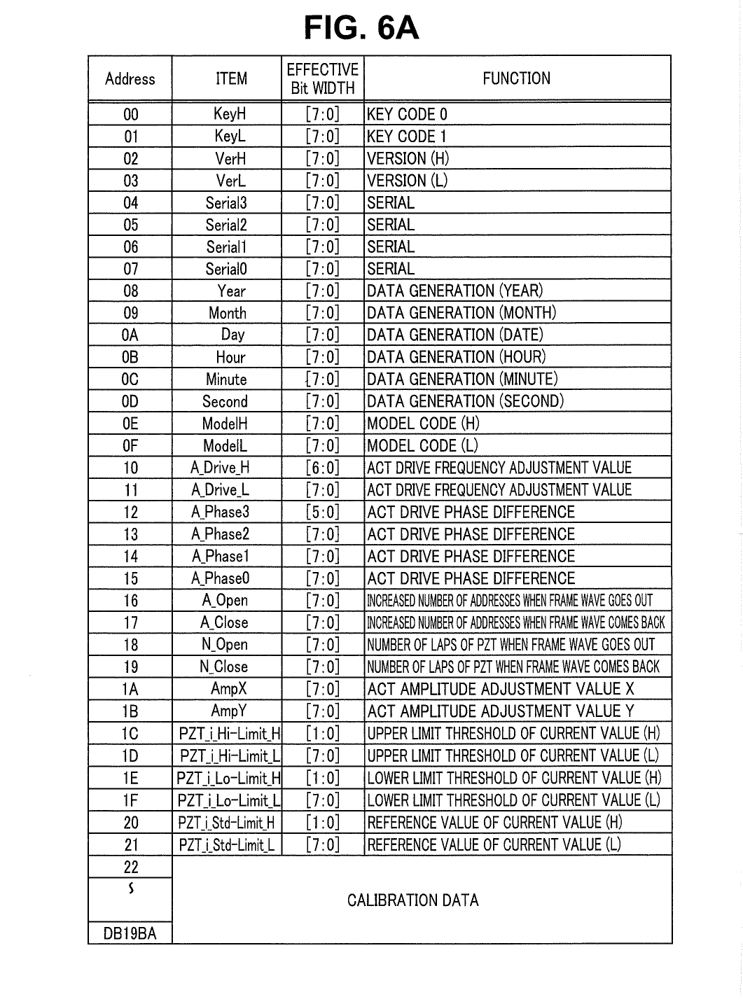

Further, the memory 6 also stores data of (information about) drive conditions such as a drive frequency for driving the actuator 16. Note that the drive conditions data is data which differs, for example, according to a type of the scanning-type endoscope 2. In a case of scanning-type endoscopes 2 of a same type, the drive conditions data may be common. FIG. 5 shows specific scope ID data (specific to the scanning-type endoscope 2) stored in the memory 6. The scope ID data is configured with a model, serial No., date of manufacture, drive conditions, various setting values and calibration data for the scanning-type endoscope 2.

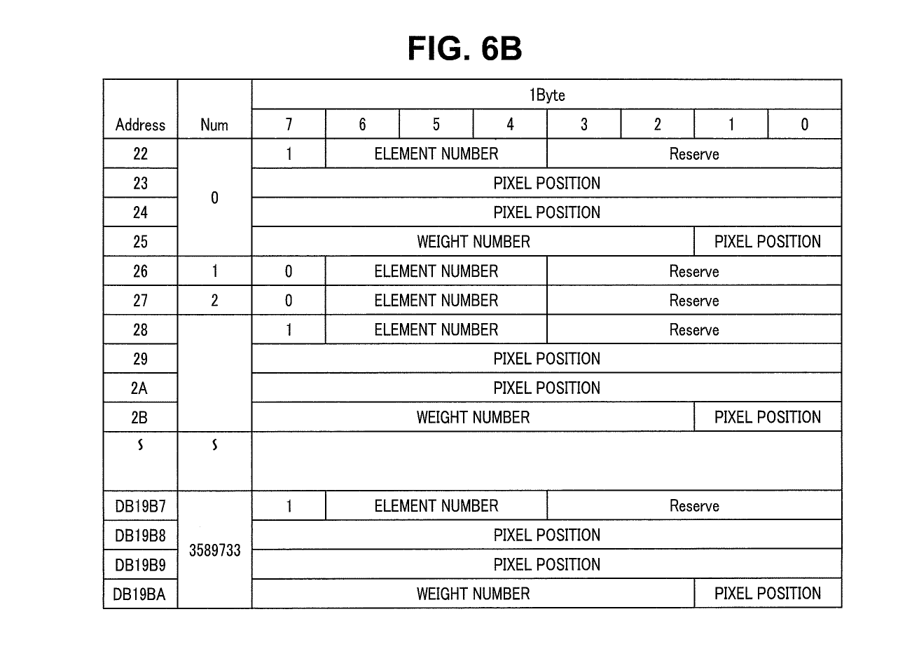

Different pieces of data of the data items are stored in memory areas associated with addresses in the memory 6 as shown in FIG. 6A. In the example shown in FIG. 6A, the drive conditions data including data of drive frequencies, phase differences and amplitude values (voltage ratios) is stored in memory areas of (memory) addresses 10 to 1B (or 0x10 to 0x1B). Further, in memory areas of addresses 1C to 21, upper limit values, lower limit values and reference values of a current which flows through the actuator 16 are stored as data of various set values.

In comparison with the data of the drive conditions and the like, data stored in memory areas of addresses 22 to DB19BA is calibration data. The calibration data is data specific to the scanning-type endoscope 2 an amount of which is much larger when compared to an amount of data of the drive conditions and the like other than the calibration data (dozens of times larger or much larger). That is, the calibration data is data which is different for each individual scanning-type endoscope 2 not only among scanning-type endoscopes 2 of different types but also among scanning-type endoscopes 2 of the same type.

Further, the calibration data is, for example, data as shown in FIG. 6B. In FIG. 6B, Num (number) indicates a position in order of light emission beginning with a center side. In data corresponding to 1 byte, flags of 0 and 1, element numbers indicating R, G and B data, a pixel position to be a scanning position, a weight number and the like are stored. Note that the pixel position is represented by 18 bits.

Since the amount of the calibration data stored in the memory 6 is much larger in comparison with the amount of the drive conditions data stored in the same memory 6, it takes much time to read the calibration data from the memory 6 via an insulation element 36c so that an image generation circuit 25b to be described later can use the calibration data at a time of generating an image. Therefore, in the present embodiment, in order that the image generation circuit 25b can use the calibration data without necessity of passing through an insulation element, preliminary or auxiliary image generation data (or image generation preset data) prepared in advance so as to be used for actuators 16 mounted on different scanning-type endoscopes in common, for example, such that is obtained by averaging a plurality of pieces of calibration data is stored, for example, in a memory 24 on the body apparatus 3 side.

The image generation circuit 25b forming an image information generating portion to be described later is arranged in a secondary circuit. In comparison, the memory 6 is arranged on a patient circuit side, the patient circuit being a circuit electrically insulated from the secondary circuit. Therefore, in order to read information stored in the memory 6 so that the information is in a state of being usable by the image generation circuit 25b, it is necessary to pass through an insulation element. Thus, to read the calibration data from the memory 6 means to read the calibration data so that the calibration data is caused to be in the state of being usable by the image generation circuit 25b arranged in the secondary circuit.

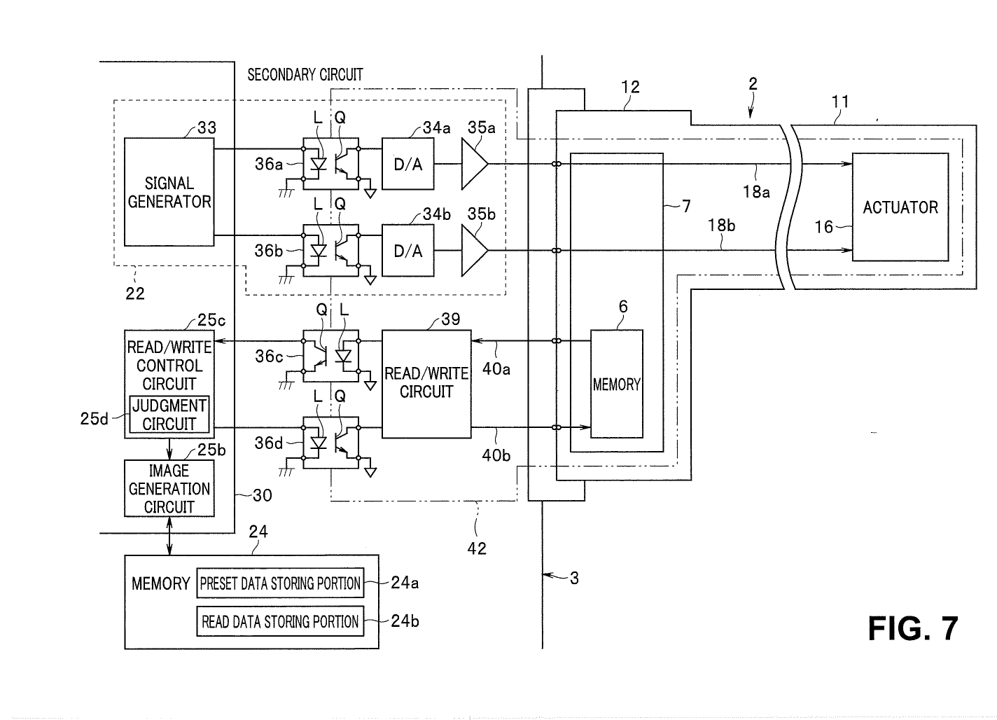

In the memory 24, the image generation preset data is stored in an image generation preset data storing portion (or an image generation preset data storing area) 24a. (Note that, in FIG. 7, the image generation preset data storing portion 24a is simply abbreviated as the preset data storing portion 24a.) Note that the memory 24 also stores information of a memory arrangement table in FIG. 6.

In a case of reading the scope ID data from the memory 6 at the time of startup when the scanning-type endoscope apparatus 1 is started up, reading of the data of the drive conditions and the like other than the calibration data is performed first so that the calibration data is read last, and, after reading of the data of the drive conditions and the like, the actuator 16 is driven to perform optical scanning. Further, the image generation preset data is used to generate an image (signal) for a detection signal acquired by the detection unit 23 when the actuator 16 is driven, and, thereby, an image at a time of performing optical scanning after startup can be displayed in a short time period.

The image generation preset data prepared in advance is not calibration data which identifies or reflects a scanning position of each individual actuator 16 with a high precision but is such calibration data that, in a case where the image generation circuit 25b in the body apparatus 3 generates an image, it is possible to identify an average scanning position in a plurality of scanning-type endoscopes 2 without requiring a waiting time period. Schematically, the image generation preset data can be said to be calibration data obtained by decreasing a precision of high-precision calibration data. Otherwise, the image generation preset data can be also said to be preliminary or auxiliary image generation information for approximating each individual piece of calibration data.

As the image generation preset data, calibration data of an average value obtained by averaging a plurality of pieces of calibration data stored in a plurality of scanning-type endoscopes 2, respectively, may be used. Therefore, by using the image generation preset data, it is possible to generate such an image signal that an amount of displacement from an accurate position is equal to or smaller than a threshold (more specifically, equal to or smaller than an amount of displacement from the average value) within a predetermined time period and with a short waiting time period and display an image of the generated image signal on the monitor 4.

In comparison, the calibration data stored in the memory 6 in each individual scanning-type endoscope 2 is calibration data which requires waiting until reading ends when the image generation circuit 25b is going to generate an image. However, the calibration data is calibration data making it possible to optimize (or identify) a scanning position of the actuators 16 mounted on each individual scanning-type endoscope 2, and, therefore, when an image is generated with use of the calibration data, it is possible to generate a high-quality image in which a scanning position by the actuator 16 of each individual scanning-type endoscope 2 is reflected with a high precision.

Therefore, as described later, when reading of the calibration data ends, the image generation circuit 25b switches use of the image generation preset data to use of the read calibration data and generates an image using the calibration data. Thereby, it is possible to generate a high-quality image as described above, and a surgeon operating the scanning-type endoscope 2 can observe the high-quality image.

As shown in FIG. 1, the body apparatus 3 is configured including: the light source unit 21 forming a light source configured to generate illumination light and supply the generated illumination light to a proximal end side of the illumination optical fiber 13 of the scanning-type endoscope 2; the drive unit 22 configured to drive the distal end of the illumination optical fiber 13 to perform scanning two-dimensionally; the detection unit 23 forming a signal generating portion configured to, using the light receiving optical fiber 15 configured to receive return light of illumination light emitted from the distal end of the illumination optical fiber 13, detect the return light, and generate a photoelectrically converted signal (or detection signal); the memory 24 to be memory areas forming a read data storing portion 24b in which the scope ID data read from the memory 6 is (temporarily) stored and to be used as a spare work area; and a controller 25 configured to control the whole body apparatus 3.

Further, the body apparatus 3 is provided with a power source circuit 26 including a secondary power source circuit 26a configured to supply a direct-current power source to each circuit arranged in the second circuit in the body apparatus 3 and a patient power source circuit 26b configured to supply a direct-current power source to each circuit arranged in a patient circuit 42 which is electrically insulated from the secondary circuit. The secondary power source circuit 26a is configured with a rectifier circuit configured to rectify an alternating-current voltage induced in secondary winding in a transformer not shown, primary winding of the transformer being connected to a commercial power source. Further, the transformer includes tertiary winding insulated from each of the primary winding and the secondary winding, and the patient power source circuit 26b is configured with a rectifier circuit configured to rectify an alternating-current voltage induced in the tertiary winding.

The light source unit 21 is configured including an R light source 31a configured to generate light of a wavelength band of red (also referred to R light), a G light source 31b configured to generate light of a wavelength band of green (also referred to G light), a B light source 31c configured to generate light of a wavelength band of blue (also referred to B light) and the multiplexer 32.

The R light source 31a, the G light source 31b and the B light source 31c are configured, for example, with laser light sources and sequentially emit R light, G light and B light, respectively, to the multiplexer 32 when being turned on by control of the controller 25. The controller 25 includes a light source control circuit 25a configured with a CPU or the like configured to control discrete light emission of the R light source 31a, the G light source 31b and the B light source 31c. When the illumination optical fiber 13 is driven by the actuator 16, the light source control circuit 25a controls light emission of the R light source 31a, the G light source 31b and the B light source 31c so as to perform pulsed light emission at driving timings for discrete coordinate positions stored in the memory 24.

Note that, in the present embodiment, the controller 25 sends control signals to cause the R light source 31a, the G light source 31b and the B light source 31c to sequentially perform pulsed light emission to the R light source 31a, the G light source 31b and the B light source 31c, and the R light source 31a, the G light source 31b and the B light source 31c sequentially generate R light, G light and B light and emit the R light, the G light and the B light to the multiplexer 32.

The multiplexer 32 sequentially supplies the R light from the R light source 31a, the G light from the G light source 31b and the B light from the B light source 31c to the light incident surface of the illumination optical fiber 13b, and the illumination optical fiber 13b sequentially supplies the R light, the G light and the B light to the illumination optical fiber 13 side.

The drive unit 22 has a function as a drive signal outputting portion and includes a signal generator 33, insulation elements 36a and 36b, D/A converters 34a and 34b and amplifiers 35a and 35b.

A drive signal generated by the drive unit 22 drives the actuator 16 mounted on the scanning-type endoscope 2 inserted in the subject 5. Therefore, a configuration is adopted in which an output signal of the signal generator 33 arranged on the secondary circuit side in FIG. 1 is electrically insulated by the insulation elements 36a and 36b as indicated by a two-dot chain line, and transmitted to the D/A converters 34a and 34b and the amplifiers 35a and 35b arranged on the patient circuit 42 side. FIG. 7 shows an electric circuit system belonging to the patient circuit 42. Note that the signal generator 33, (the image generation circuit 25b in) the controller 25, the memory 24 and the like except the patient circuit 42 in the body apparatus 3 in FIG. 1 belong to the secondary circuit or arranged in the secondary circuit.

The signal generator 33 generates a drive signal for causing the end portion of the illumination optical fiber 13 which includes the light emission surface to be vibrated (or swung) based on control of the controller 25 and outputs the drive signal to the D/A converters 34a and 34b via the insulation elements 36a and 36b.

As shown in FIG. 7, by being configured with a light emitting diode (abbreviated as an LED) L and a phototransistor Q and configured to convert an electric signal to an optical signal and further convert an optical signal to an electric signal (at an optical coupling portion), each of the insulation elements 36a and 36b (additionally, insulation elements 36c and 36d to be described later) electrically insulates the secondary circuit side to be a signal input side of the optical coupling portion and the patient circuit 42 side to be a signal output side.

Note that, in FIG. 1, the controller 25 and the signal generator 33 are configured with a programmable semiconductor such as an FPGA (field programmable gate array) 30 as indicated by a two-dot chain line. Note that a part of the controller 25 may be configured with a central processing unit (CPU).

The D/A converters 34a and 34b convert digital drive signals outputted from the signal generator 33 to analog drive signals and output the analog drive signals to the amplifiers 35a and 35b, respectively.

The amplifiers 35a and 35b amplify the drive signals with a small amplitude outputted from the D/A converters 34a and 34b, respectively, to cause the drive signals to be the drive signals shown in FIG. 3 and output the drive signals to the actuator 16. Note that a power source for operation is supplied to the D/A converters 34a and 34b, the amplifiers 35a and 35b and the like belonging to the patient circuit 42 from the patient power source circuit 26b insulated from the secondary power source circuit 26a.

The detection unit 23 in the body apparatus 3 includes the detector 37 and an A/D converter 38.

The detector 37 is configured with a photodetector such as a photodiode and configured to receive return light emitted from the light emission surface of the light receiving optical fiber 15 and converts the return light to an electric signal. The light receiving optical fiber 15 guides return lights reflected by the subject 5 illuminated sequentially by R light, G light and B light, and the guided return lights are sequentially caused to be incident to the detector 37. The detector 37 sequentially generates analog R, G and B detection signals according to intensities of the incident R, G and B return lights and outputs the analog R, G and B detection signals to the A/D converter 38.

The A/D converter 38 sequentially converts the analog R, G and B detection signals sequentially outputted from the detector 37 to digital R, G and B detection signals, respectively, and outputs the digital R, G and B detection signals to the image generation circuit 25b in the controller 25. Note that, in the configuration shown in FIG. 1, the R light source 31a, the G light source 31b and the B light source 31c may be configured to perform pulsed light emission at the same time. In this case, the detection unit 23 can be configured to detect R light, G light and B light at the same time.

In the memory 24, a control program for controlling the body apparatus 3 and the like are stored in advance. Further, in a part of memory areas of the memory 24, the scope ID data read from the memory 6 by the controller 25 of the body apparatus 3 is stored. For example, the part of the memory areas in the memory 24 form the read data storing portion 24b configured to temporarily store the scope ID data read from the memory 6. In the read data storing portion 24b, the drive conditions data and the calibration data described above are stored.

For example, the controller 25 controls the light source unit 21, the drive unit 22 and the like based on the control program and the like stored in the memory 24.

The actuator 16 having a function as a scanner causes the illumination optical fiber 13 to be swung so that a trajectory corresponding to a predetermined scanning pattern in which a radiation position of illumination light radiated to an object forms a spiral shape, based on a drive signal outputted from the drive unit 22 in response to control of the controller 25 as described above.

Further, the light source control circuit 25a of the controller 25 performs control to cause the R light source 31a, the G light source 31b and the B light source 31c to sequentially and discretely emit light, in accordance with information about light emission positions (or light emission timings) associated with drive signals stored in the memory 24 in advance. Then, the detection unit 23 samples and acquires return lights from an object as R, G and B detection signals at timings of light emissions sequentially performed and stores the acquired R, G and B detection signals into a memory in the image generation circuit 25b forming an image generating portion or the image information generating portion.

Further, a read/write control circuit 25c provided in the controller 25 is connected to a read/write circuit 39 provided on the patient circuit 42 side via the insulation elements 36c and 36d, and the read/write circuit 39 is connected to the memory 6 via signal lines 40a and 40b. The read/write control circuit 25c controls the read/write circuit 39 to read the scope ID data from the memory 6 at startup. The read scope ID data is outputted to the memory 24 (arranged in the common secondary circuit) which can be accessed from the image generation circuit 25b forming the image information generating portion and the like via the read/write control circuit 25c and the insulation element 36c.

Note that the image generation circuit 25b may control the read/write circuit 39 to acquire the scope ID data read from the memory 6.

The read/write control circuit 25c includes a judgment circuit 25d configured to judge whether the data read via the read/write circuit 39 is data having an error or not. The judgment circuit 25d is not limited to the case of being provided inside the read/write control circuit 25c. For example, a configuration may be adopted in which the read/write circuit 39 or the image generation circuit 25b is provided with the judgment circuit 25d.

As described later, if the read data has an error, the same data is read, for example, several times.

It is also conceivable that a user such as a surgeon inputs a set value Ns for the number of times of repeatedly reading same data from an input device 43 configured with a keyboard and the like in advance, and, if read data has an error, the same data is read the number of times equal to or smaller than the inputted set value Ns for the number of times. As the set value Ns, a natural number of at least 2 or more is set.

In the present embodiment, when the scope ID data is read from the memory 6, the data arrangement table in the memory 6 provided in the body apparatus 3 in advance is referred to, and data other than the calibration data is read prior to the calibration data. Then, when reading of the drive conditions data ends, the drive unit 22 generates drive signals by referring to the read drive conditions data, and the light source control circuit 25a starts control to cause the R light source 31a, the G light source 31b and the B light source 31c to emit light in conjunction with the drive signals.

Further, return light from the subject 5 is detected by the detection unit 23, and a detected detection signal is inputted to the image generation circuit 25b.

The image generation circuit 25b identifies a two-dimensional position on an image of the detection signal, using the image generation preset data for the detection signal, converts the identified two-dimensional position to a raster scan position to generate an image signal, and outputs the generated image signal to the monitor 4.

Further, after reading of the calibration data ends, the image generation circuit 25b identifies a two-dimensional position on the image of the detection signal, not using the image generation preset data but using the read calibration data, for the detection signal, converts the identified two-dimensional position to a raster scan position to generate an image signal, and outputs the generated image signal to the monitor 4.

Note that the controller 25, the light source control circuit 25a, the image generation circuit 25b, the read/write control circuit 25c and the judgment circuit 25d are not limited to the case of being configured with the CPU or the FPGA described above but may be configured with dedicated hardware.

The scanning-type endoscope apparatus 1 of the present embodiment is characterized in including: the scanning-type endoscope 2 configured to, by driving the actuator 16 for swinging a fiber configured to guide illumination light radiated to the subject 5, perform scanning with the illumination light on the subject 5; the memory 6 forming a storing portion provided in the scanning-type endoscope 2 and configured to store the drive conditions information which is information about the drive conditions of the actuator 16 and the calibration data as specific information which is information specific to the scanning-type endoscope 2, the specific information having a larger amount of data than the drive conditions information; the read/write control circuit 25c and the read/write circuit 39 forming a reading portion configured to read the drive conditions information prior to the information specific to the scanning-type endoscope 2 among pieces of information stored in the storing portion; and the controller 25 forming a controlling portion configured to perform control to drive the actuator 16 based on the drive conditions information read by the reading portion first. Note that the scanning-type endoscope apparatus 1 may be configures so that the reading portion is configured further including the insulation element 36c.

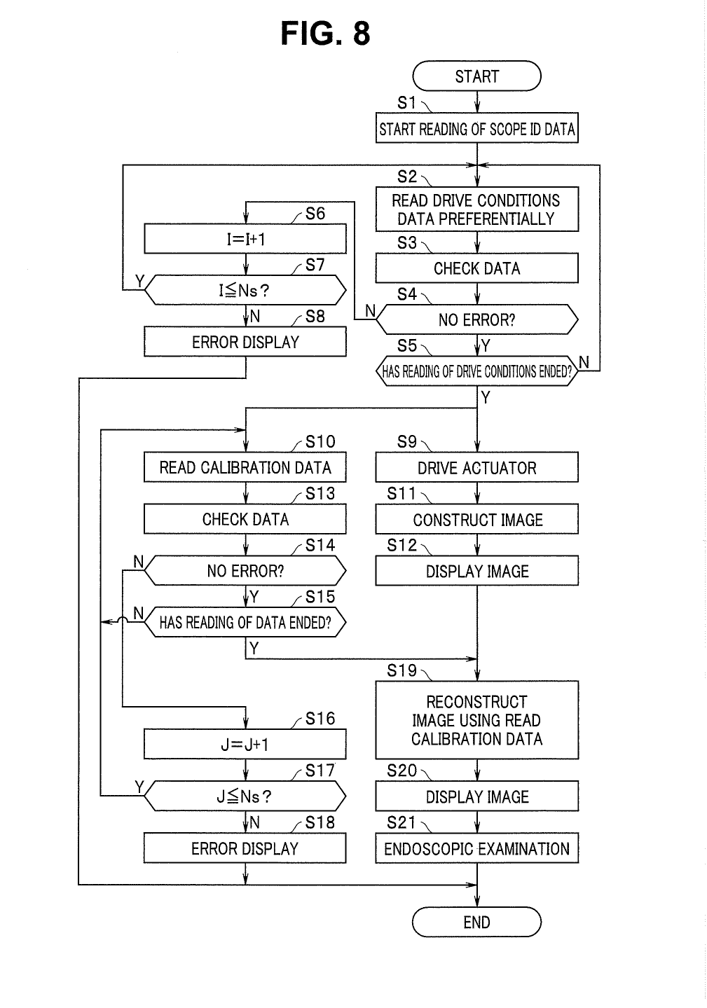

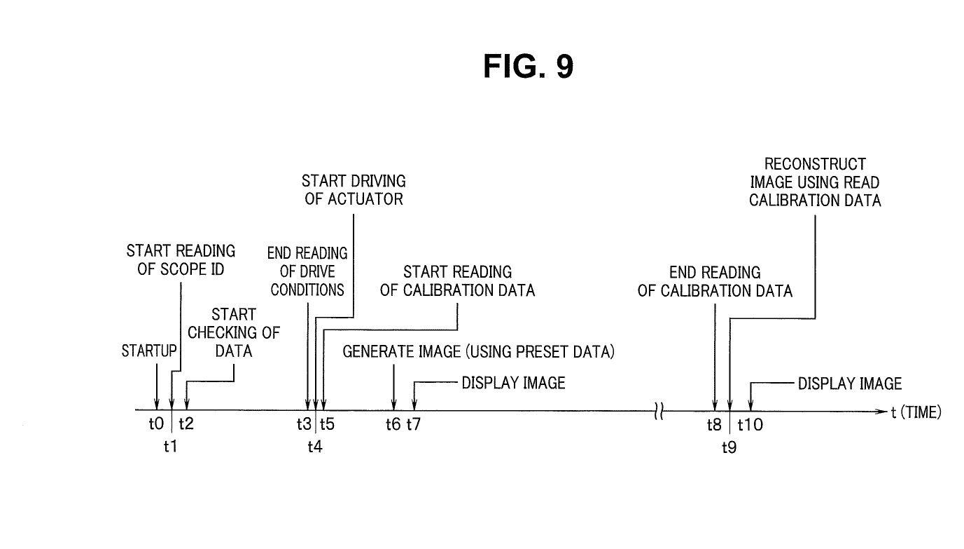

Next, operation of the present embodiment will be described with reference to FIGS. 8 and 9. FIG. 8 shows a process procedure in a case of typical operation of the present embodiment; and FIG. 9 shows temporal timings of the typical operation of the present embodiment.

When the scanning-type endoscope 2 is connected to the body apparatus 3 and powered on, each circuit in the body apparatus 3 enters an operation state. When the body apparatus 3 is started up (in FIG. 9, startup time is indicated by time t0), (the read/write control circuit 25c of) the controller 25 starts reading of the scope ID data from the memory 6 at step S1. In FIG. 9, time of starting reading of the scope ID data is indicated by t1. In this case, as shown in step S2, (the read/write control circuit 25c of) the controller 25 refers to the memory arrangement table and starts reading in a reading state in which the data of the drive conditions and each set value is read prior to the calibration data (in FIG. 8, written as “read drive conditions data preferentially”).

As shown in step S3, (the judgment circuit 25d of) the read/write control circuit 25c checks the data read from the memory 6. In FIG. 9, time of starting the data checking is indicated by t2. Note that the data checking is performed until reading of the scope ID data ends.

The following is a data checking method. A binary numerical value is separated into certain units, and data is transmitted, with 1-bit numerical value attached to an end of each unit so that the number of 1's included in each unit is necessarily an even number or an odd number. Then, parity check for confirmation on a receiving side (a reading side), check sum and the like are performed, the check sum being such that, by dividing the data before transmission, calculating a total value by regarding pieces of data in respective blocks as numerical values, and transmitting the total value together with the data, checking is performed on a receiving side.

At next step S4, (the judgment circuit 25d of) the read/write control circuit 25c judges whether or not there is not an error (data is correct). If the is not an error, the flow proceeds to next step S5.

On the other hand, in a case of a judgment result that there is an error, (the judgment circuit 25d of) the read/write control circuit 25c sets a parameter I of the number of repetitions to I=I+1, which is a value obtained by adding 1 to an initial value 0, at step S6, and, after that, judges whether or not I is equal to or smaller than the set value Ns at step S7. Since I is 1 currently, it is judged that I is equal to or smaller than the set value Ns. The flow returns to the process of step S2, and a similar process is repeated.

In general, reading can be normally performed by repeatedly reading data including an error for a number of times equal to or smaller than the set value Ns. However, if reading of data without an error cannot be performed by the number of times of repetition equal to or smaller than the set value Ns, an error display is displayed on the monitor 4 at step 8, and the process of FIG. 8 ends.

At step S5, (the read/write control circuit 25c of) the controller 25 judges whether reading of data (up to) the drive conditions has ended or not. In a case of a judgment result that reading of the data (up to) the drive conditions has not ended, the flow returns to the process of step S2, and a similar process is repeated.

On the other hand, in a case of a judgment result that reading of the data (up to) the drive conditions has ended, the flow proceeds to a process of step S9. In FIG. 9, time at which reading of the data (up to) the drive conditions ends (is completed) is indicated by t3.

At step S9, the drive unit 22 and (the light source control circuit 25a of) the controller 25 refer to the drive conditions data which has been read and perform (start operation of) driving of the actuator 16 and light emission control of the light sources in conjunction. In FIG. 9, time of starting driving of the actuator 16 is indicated by t4. Further, when reading of the data (up to) the drive conditions ends, the read/write control circuit 25c starts reading of the calibration data as shown in step S10. In FIG. 9, time of starting reading the calibration data is indicated by t5. The processes of step S9 and S10 are performed in parallel.

The actuator 16 is driven; return light from an object at a time of causing the light sources to emit light is detected by the detection unit 23; and a detection signal is inputted to the image generation circuit 25b. Note that, at this stage, the scanning-type endoscope 2 is not inserted in the subject 5 yet.

As shown in step S11, the image generation circuit 25b generates an image signal (constructs an image) using the image generation preset data stored in the memory 24 each time a detection signal corresponding to one frame is acquired. Further, as shown in step S12, the image generation circuit 25b outputs the generated image signal to the monitor 4, and the monitor 4 displays the generated image.

In FIG. 9, for example, time of starting first image generation is indicated by t6, and time of first image display is indicated by t7. Thus, by using the image generation preset data, the surgeon can confirm a first image at the time t7 with a relatively short waiting time period (t740) from the startup time t0. Though the image in this case is an image obtained by optical scanning outside a body, it is possible to confirm whether or not an image can be obtained by optical scanning, in a short time period.

On the other hand, (the judgment circuit 25d of) the read/write control circuit 25c which has started reading of the calibration data at step S10 checks the read calibration data at step S13. At next step S14, (the judgment circuit 25d of) the read/write control circuit 25c judges whether or not the read calibration data does not have an error (the data is correct). If the calibration data does not have an error, the flow proceeds to a process of next step S15.

On the other hand, in a case of a judgment result that the calibration data has an error, (the judgment circuit 25d of) the read/write control circuit 25c sets a parameter J of the number of repetitions in the case of calibration data to J=J+1, which is a value obtained by adding 1 to an initial value 0, at step S16, and, after that, judges whether or not J is equal to or smaller than the set value Ns at step S17. Since J is 1 currently, it is judged that J is equal to or smaller than the set value Ns. The flow returns to the process of step S10, and a similar process is repeated.

In general, reading can be normally performed by repeatedly reading data including an error for a number of times equal to or smaller than the set value Ns. However, if reading of data without an error cannot be performed by the number of times of repetition equal to or smaller than the set value Ns, an error display is performed on the monitor 4 at step S18, and the process of FIG. 8 ends.

At step 15, the read/write control circuit 25c judges whether or not reading of the calibration data has ended (or has been completed). If reading of the calibration data has not ended, the flow returns to the process of step S10. On the other hand, in a case of a judgment result that reading of the calibration data has ended, a signal indicating that reading of the calibration data has ended is transmitted to the image generation circuit 25b. In FIG. 9, time at which reading of the calibration data ends is indicated by t8.

The image generation circuit 25b which has received the signal indicating that reading of the calibration data has ended performs image reconstruction using the calibration data as shown in step S19.

Before image reconstruction is performed, image construction has been performed with use of the image generation preset data when a detection signal is acquired. After the time when the signal indicating that reading of the calibration data has ended is received, an image is constructed (generated) not with use of the image generation preset data but with use of the calibration data for an acquired detection signal, and a constructed image signal is outputted to the monitor 4. To construct (generate) an image using calibration data is also referred to image reconstruction. In FIG. 9, time of starting image reconstruction using the calibration data is indicated by t9.

At next step S20, the monitor 4 displays the constructed image using the calibration data. In FIG. 9, time of displaying the image using the calibration data is indicated by t10. Note that waiting time t1040 from the startup time t0 to the time t10 of displaying the image using the calibration data is several times as long as the waiting time t7-t0 described above.

After the state of constructing (generating) an image using the calibration data is entered, the surgeon inserts the scanning-type endoscope 2 into the subject 5 to perform an endoscopic examination as shown in step S21. Thus, the process of FIG. 8 ends.

According to the present embodiment which operates as described above, though, at the time of reading the scope ID data from the memory 6 of the scanning-type endoscope 2 connected to the body apparatus 3 via the insulation element 36c at startup time, it takes much time to read the calibration data, it is possible to, by reading the drive conditions data first, driving the actuator 16 immediately after the reading to acquire a detection signal, and identifying a radiation position of the acquired detection signal using the preliminary image generation preset data prepared in advance, display an optical scan image in a short time with a short waiting time from the startup time. Therefore, the surgeon can confirm an image acquired by the scanning-type endoscope 2 after the short waiting time after the startup time, and it is possible to improve operability for the surgeon.

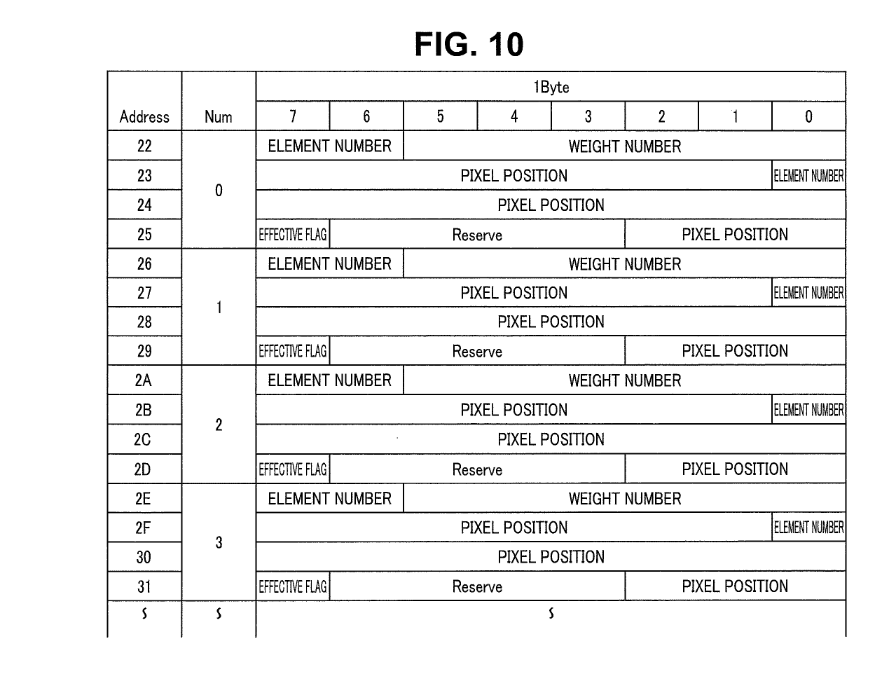

Note that an amount of the calibration data shown in FIG. 6B has been reduced, for example, in comparison with an amount of data as shown in FIG. 10 so that a time period required for reading is shortened.

To make a supplementary description, the calibration data stored in the memory 6 of the scanning-type endoscope 2 is periodically corrected and updated with use of the body apparatus 3. Then, the calibration data to be updated is temporarily stored in the memory 24 of the body apparatus 3 and written to the memory 6 under control of the read/write control circuit 25c.

Since the memory on the body apparatus 3 side such as the memory 24, which stores the calibration data, can perform a read/write process at a high speed not via the insulation element 36c and the like, such a waiting time period that a user such as a surgeon feels long does not happen even if unnecessary data exists in the calibration data.

However, since the calibration data stored in the memory 6 provided in the scanning-type endoscope 2 is required to be read via the insulation element 36c, speed of reading the calibration data is slower in comparison with the case of reading not via the insulation element 36c. Therefore, the waiting time period which a user such as a surgeon feels long occurs.

Therefore, as for the calibration data stored in the memory 6 provided in the scanning-type endoscope 2, the waiting time period can be reduced by deleting unnecessary data as much as possible to reduce the amount of data. In the present embodiment, if the calibration data updated on the body apparatus 3 side is, for example, such that is as shown in FIG. 10, the amount of data is reduced before writing to the memory 6 as shown in FIG. 6B. By storing the reduced calibration data into the memory 6 as described above, it is possible to, in the case of performing an endoscopic examination using the scanning-type endoscope 2 connected to the body apparatus 3, shorten the time of reading the calibration data from the memory 6 at the startup time.

Note that, in the description above, the actuator 16 may be driven after reading the data of “format” up to “various set values” in FIG. 5 excluding the calibration data in the scope ID data.

Further, since the calibration data and the like can be read and written from the read data storing portion 24b provided in the body apparatus 3 at a high speed not via an insulation element on the body apparatus 3 side, the merit may be used as shown in a modification below.

For each scanning-type endoscope 2, if calibration data for the actuator 16 mounted on the scanning-type endoscope 2 is updated, data of (information about) a latest date of update (date of update) is recorded (stored). For example, data of a date of manufacture is recorded so as to include data of a date of update.

Further, in a storage device configured with the read data storing portion 24b and the like provided in (the secondary circuit of) the body apparatus 3 (though the storage device is the memory 24 in FIGS. 1 and 7, the storage device is not limited to the memory 24), calibration data and dates of update read from memories 6 of scanning-type endoscopes 2 which have been connected to the body apparatus 3 corresponding to storage capacity of the storage device is stored, for example, being associated with serial Nos. to be identification information about the scanning-type endoscopes 2. In this case, the other data (models, drive conditions, various set values) shown in FIG. 5 may be stored being associated with the serial Nos. Note that it is recommended to, in a case of storing (recording) calibration data, dates of update and serial Nos. exceeding the storage capacity, store the data by preferentially overwriting an area of temporally oldest data.

When a scanning-type endoscope 2 is connected to the body apparatus 3, and at least data of a serial No. and a date of update is read from the memory 6 of the scanning-type endoscope 2, the body apparatus 3 judges whether the storage device on the body apparatus 3 side stores the same data or not, and, if the same data is stored, constructs an image using the calibration data stored in the storage device on the body apparatus 3 side. In this case, the image is constructed not with use of the image generation preset data but with use of the calibration data.

On the other hand, if the same data is not stored, an image is generated as in the first embodiment described above.

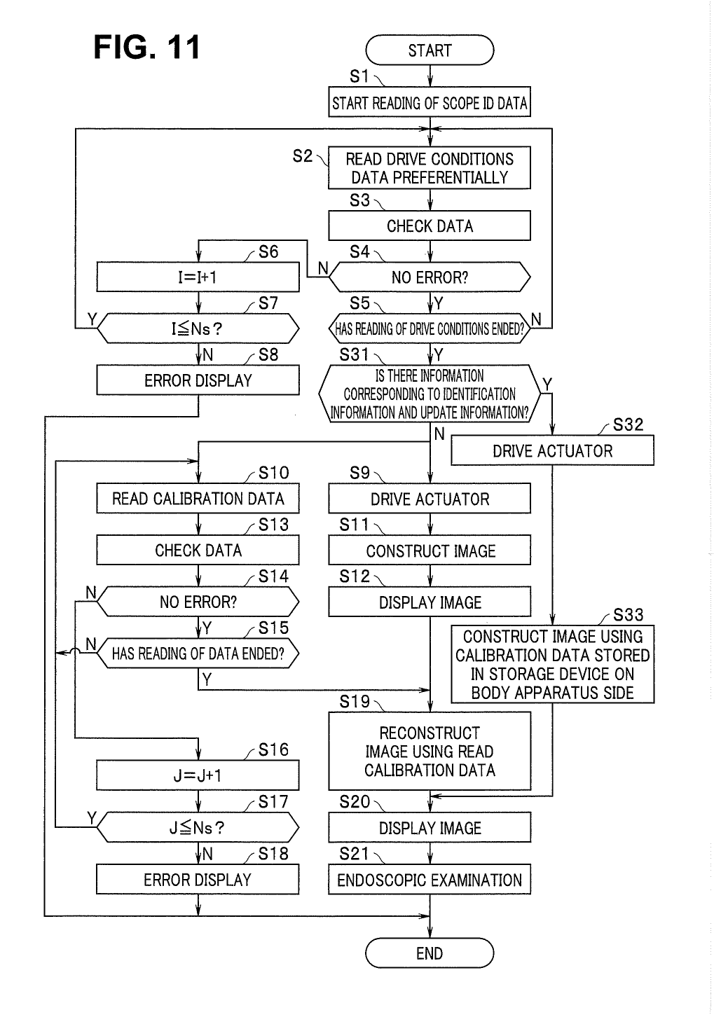

FIG. 11 shows a flowchart of a process example in this case. For example, the process shown in FIG. 11 has step S31 for a process of performing judgment and steps S32 and S33 to be performed according to a judgment result of S31 between step S5 and steps S9 and S10 in the process in FIG. 8. The process shown in FIG. 11 is the same process as shown in FIG. 8 except for steps S31 to S33.

If the scope ID data is read as described above, the read/write control circuit 25c (or the judgment circuit 25d) judges whether or not the storage device on the body apparatus 3 side stores information corresponding to the identification information and the date of update information, which is the latest update information, at step S31. Note that the memory 6 stores, together with the calibration data to be stored, date and time information about a date of creation of the calibration data. Further, when old calibration data is updated, the memory 6 is updated (overwritten) with updated new calibration data and date and time information about a date of the update.

If the corresponding information is not stored as a result of the above judgment, the processes of steps S9 and S10 are performed.

In comparison, if it is judged that the corresponding information is stored, the flow proceeds to a process of next step S32, and the actuator 16 is driven similar to step S9. At step S33, for a detection signal acquired at the time of driving the actuator 16, the image generation circuit 25b performs the process for constructing (generating) an image using the calibration data stored in the storage device on the body apparatus 3 side.

Then, the generated image is displayed on the monitor 4 at step S20. In this case, the process for generating an image using the preliminary calibration data in FIG. 8 is not performed, but a high-quality image is generated in a short time period with use of calibration data corresponding to the actuator 16 mounted on the connected scanning-type endoscope 2.

Since the operation described above is performed, there is an advantage that, in a case where, after an endoscopic examination is performed with use of a scanning-type endoscope 2 connected to the body apparatus 3, an endoscopic examination is performed again with use of the same the scanning-type endoscope 2 before a lot of days elapse, it is possible to display a high-quality image after a short waiting time period.

Note that a part of the present embodiment and the like may be omitted to configure a scanning-type endoscope apparatus. For example, a read control circuit and a read circuit having functions related to reading of the read/write control circuit 25c and the read/write circuit 39 may be used to configure a scanning-type endoscope apparatus. Further, a configuration may be adopted in which the read/write control circuit 25c or the read control circuit can read the scope ID data from the memory 6 via the insulation element 36c. Further, a configuration may be adopted in which the scope ID data can be read from the memory 6 with use of a plurality of insulation elements as the insulation element 36c.

Note that, though an example has been described in which reading of the data of the drive conditions and the like other than the calibration data in the scope ID data is performed prior to reading of the calibration data while data checking is performed in the embodiment and the like described above, the actuator may be driven after all the scope ID data including the calibration data is read without an error.

What is claimed is:

1. A scanning-type endoscope apparatus comprising:
a scanning-type endoscope configured to, by driving an actuator for swinging a fiber configured to guide illumination light radiated to a subject, perform scanning with the illumination light on the subject;
a storing portion provided in the scanning-type endoscope and configured to store drive conditions information which is information about drive conditions of the actuator and specific information which is information specific to the scanning-type endoscope, the specific information having a larger amount of data than the drive conditions information and being about the scanning;
a reading portion configured to read the drive conditions information and the specific information among pieces of information stored in the storing portion;
a controlling portion configured to perform control to drive the actuator based on the drive conditions information read by the reading portion;
a body apparatus to which the scanning-type endoscope is detachably connected, the body apparatus comprising the reading portion and the controlling portion;
a signal generating portion provided in the body apparatus and configured to swing the fiber using the actuator driven in accordance with the drive conditions information read by the reading portion, receive light from the subject at a time of radiating the illumination light, and generate a photoelectrically converted signal; and
an image information generating portion provided in the body apparatus and configured to generate image information corresponding to a radiation position of the illumination light based on the signal; wherein
in a first state in which reading of the drive conditions information from the storing portion by the reading portion is complete but reading of the specific information is not complete, the controlling portion controls the image information generating portion to generate the image information to be displayed on a display apparatus using at least the signal generated by the signal generating portion; and
in a second state in which reading of the drive conditions information and the specific information from the storing portion by the reading portion is complete, the controlling portion controls the image information generating portion to generate the image information to be displayed on the display apparatus using both of the signal generated by the signal generating portion and the specific information read by the reading portion.

2. The scanning-type endoscope apparatus according to claim 1, further comprising an image generation information storing portion provided in the body apparatus and configured to store preliminary image generation information prepared in advance in order for the image information generating portion to generate the image information to be displayed on the display apparatus from the signal generated by the signal generating portion, wherein
in the first state, the controlling portion controls the image information generating portion to generate the image information to be displayed on the display apparatus using the preliminary image generation information.

3. The scanning-type endoscope apparatus according to claim 1, wherein
the reading portion reads the specific information different from the drive conditions information after reading the drive conditions information; and
the controlling portion performs control to drive the actuator at a timing of the first state in which the reading portion reads the specific information.

4. The scanning-type endoscope apparatus according to claim 1, further comprising a judging portion configured to judge whether the drive conditions information read by the reading portion is normal or abnormal, wherein
the controlling portion performs control to drive the actuator based on the drive conditions information if the judging portion judges that the drive conditions information is normal and performs control not to drive the actuator if the judging portion judges that the drive conditions information is abnormal.

5. The scanning-type endoscope apparatus according to claim 1, wherein the storing portion is configured to store image generation information used at the time of the image information generating portion generating the image information about the subject from the signal generated by the signal generating portion, as the specific information.

6. The scanning-type endoscope apparatus according to claim 2, wherein, during a period of the second state after reading of the drive conditions information and the specific information by the reading portion is complete and in the case of generating the image information to be displayed on the display apparatus from the signal generated by the signal generating portion, the controlling portion performs control to switch from use of the preliminary image generation information used in the first state in which reading of the specific information is not ended to use of the specific information to generate the image information to be displayed on the display apparatus.

7. The scanning-type endoscope apparatus according to claim 1, further comprising:
an image generation information storing portion provided in the body apparatus and configured to store preliminary image generation information prepared in advance in order to generate the image information to be displayed on the display apparatus from the signal generated by the signal generating portion; and
a read information storing portion provided in the body apparatus and configured to store the drive conditions information, the specific information and latest date and time information about a date and time of creation or update of the specific information read from the storage portion with identification information identifying the scanning-type endoscope; wherein
the controlling portion judges whether or not the read information storing portion stores information corresponding to the identification information and the latest date and time information read from the storing portion by the reading portion, and, if the corresponding information is stored, performs control to generate the image information to be displayed on the display apparatus using second specific information corresponding to the specific information stored in the read information storing portion.

8. The scanning-type endoscope apparatus according to claim 2, wherein the image generation information storing portion stores average specific information obtained by averaging the specific information in plurality stored in the storing portion in plurality of a plurality of scanning-type endoscopes connectable to the body apparatus, including the scanning-type endoscope, as the preliminary image generation information.

9. The scanning-type endoscope apparatus according to claim 2, wherein
the storing portion stores calibration data for calibrating the radiation position of the illumination light radiated on the subject at the time of swinging the fiber using the actuator as the specific information; and
the image generation information storing portion stores average calibration data obtained by averaging the calibration data in plurality stored in the storing portion in plurality of a plurality of scanning-type endoscopes connectable to the body apparatus including the scanning-type endoscope, as the preliminary image generation information.

10. The scanning-type endoscope apparatus according to claim 9, wherein, during a period of the second state after reading of the drive conditions information and the specific information by the reading portion is complete, the controlling portion controls the image information generating portion to stop using the average calibration data and generate the image information to be displayed on the display apparatus using the calibration data as the specific information read by the reading portion.

11. The scanning-type endoscope apparatus according to claim 2, further comprising a secondary circuit in which the signal generating portion, the image information generating portion and the controlling portion are arranged in the body apparatus; and
a second storing portion configured to store the specific information read by the reading portion from the storing portion electrically insulated from the secondary circuit is provided in the secondary circuit.

12. The scanning-type endoscope apparatus according to claim 3, further comprising an image generation information storing portion configured to store auxiliary image generation information for approximating the specific information; wherein
at timings in the first state in which the reading portion reads the specific information, the control portion performs control to drive the actuator to perform scanning with the illumination light and control to generate an image corresponding to the scanning with the illumination light using the auxiliary image generation information in parallel.

13. The scanning-type endoscope apparatus according to claim 11, further comprising a judging portion configured to judge whether or not the second storing portion provided in the body apparatus stores second information which is a same as the specific information stored in the storing portion of the scanning-type endoscope connected to the body apparatus, in the body apparatus; wherein
if it is judged by the judging portion that the second information which is the same as the specific information is stored, the controlling portion controls the image information generating portion to generate the image information to be displayed on the display apparatus not using the preliminary image generation information but using the second information.
